(19) United States
(12) Patent Application Publication
(10) Pub. No.: US 2010/0292652 A1
(43) Pub. Date: Nov. 18, 2010

(54) NEEDLE CAP

(76) Inventor: Denis Gantin, Bonneville (FR)

Correspondence Address: ALLERGAN, INC., 2525 DUPONT DRIVE, T2-7H, IRVINE, CA, 92612-1599, US

(21) Appl. No.: 12/305,945

(22) PCT Filed: Jul. 3, 2007
(86) PCT No.: PCT/FR07/51586

§ 371 Date: Jan. 13, 2010

(30) Foreign Application Priority Data
Jul. 4, 2006 (FR) 0652786

(51) Int. Cl. A61M 5/32

(52) U.S. Cl. 604/192

(57) ABSTRACT

The present invention pertains to a cap (40) for a syringe needle designed to be mounted on the body of a syringe. The cap is characterized in that it comprises:
an envelope-forming part (42) for receiving the needle;
a fixation piece (44) capable of receiving the base of the needle and comprising on its external surface a threaded section (54) capable of cooperating with an internal screw thread arranged at one end of the syringe body; and
divisible portions (46a, 46b) for connecting the said envelope-forming part and the said fixation piece, the divisible portions having certain dimensions so that they break when a predetermined torque is applied to the envelope-forming part, even though the threaded section of the fixation piece was engaged with the internal screw thread of the syringe body.

The object of the present invention is a needle cap for a syringe and a syringe equipped with the said cap.

More precisely, the present invention pertains to a cap for a syringe needle, which makes it possible to improve the fixation of the needle on the syringe body.

The present invention pertains more particularly to a type of syringe used especially, but not exclusively, in the area of aesthetic medicine, in dermatology and especially for the subcutaneous injection of products capable of remodeling the appearance of the skin.

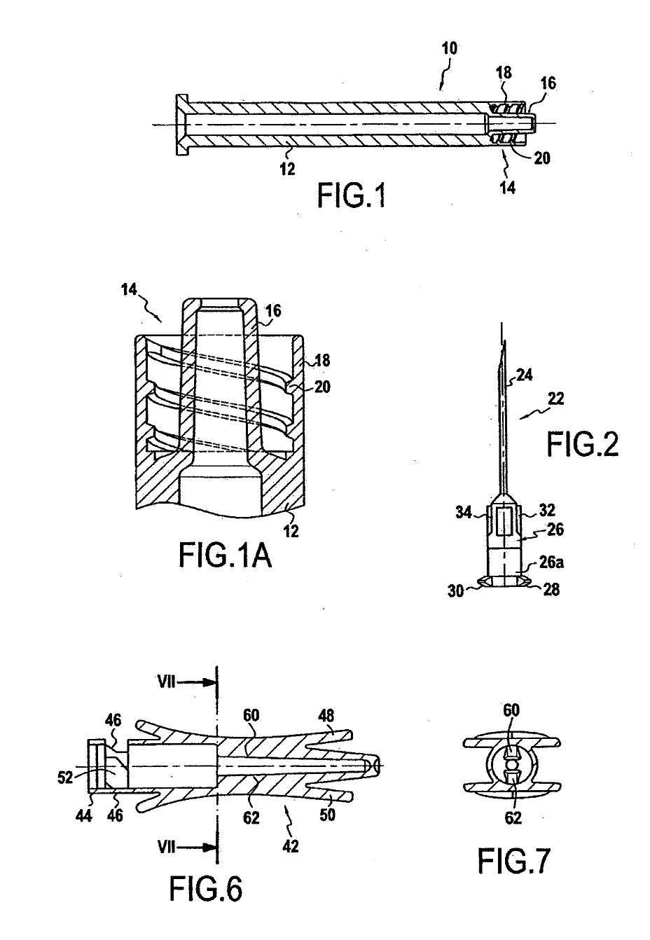

The present invention applies especially to a syringe and to a needle assembly of the type shown in FIGS. 1, 1A and 2.

The syringe 10 shown in FIGS. 1 and 1A classically comprises a cylindrical body 12 having a fixation end 14. The fixation end 14 consists of a centering truncated cone 16 and of a ferrule 18 surrounding the cone 16 and provided with an internal screw thread 20.

FIG. 2 shows the needle assembly 22, which consists of the needle proper 24 and a hollow base 26. At its end 26a opposite to the needle, the base is provided with two radial projections 28 and 30, which form projections capable of cooperating with the internal screw thread 20 of the ferrule 18 of the syringe body 12. The two radial projections constitute a threaded portion. At its other end, the base 26 preferably comprises two fins 32 and 34 whose function shall be explained later.

To put the needle assembly 22 in place on the syringe 10, the internal passage of the base 26 is engaged on the end of the truncated cone 16 and the radial extensions 28 and 30 cooperate with the internal screw thread 20 of the syringe body. By turning the base of the needle, a screwing of the needle on the syringe body is obtained.

Experience shows that in the case in which the liquid that has to be injected and [sic, typo for “est”—is—Tr.Ed.] relatively fluid, the mode of fixing the needle on the syringe body is entirely satisfactory. On the other hand, when the liquid to be injected has a higher viscosity, this mode of connection often proves to be insufficient, especially if, during the mounting of the needle on the syringe body, the user does not sufficiently screw the base on the syringe body. Such a situation occurs especially, but not exclusively, when one wants to inject under the skin of the patient compounds based on hyaluronic acid, whose viscosity is relatively high.

A first object of the present invention is to provide a needle cap for a syringe, especially of the type described in relation to FIGS. 1, 1A and 2, which not only protects the needle but also makes it possible to ensure a highly reliable fixation of the needle on the syringe body.

To accomplish this object according to the present invention, the cap for a syringe needle designed to be mounted on the body of a syringe is characterized in that it comprises:
an envelope-forming part for receiving the needle;
a fixation piece capable of receiving the base of the needle and comprising on its external surface a threaded section capable of cooperating with an internal screw thread arranged at one end of the syringe body; and
divisible portions for connecting the said envelope-forming part and the said fixation piece, the divisible portions having certain dimensions so that they break when a predetermined torque is applied to the envelope-forming part, even though the threaded section of the fixation piece was engaged with the internal screw thread of the syringe body.

It is understood that the divisible portions are determined in such a way that they are broken when the screwing torque of the cap on the syringe body is sufficient. Thus, the fixation piece remains screwed in the internal screw thread of the syringe body, even though the envelope-forming part of the cap is released. The fixation piece, which cooperates with the internal screw thread, is the equivalent of a blocking locknut, thus ensuring a highly reliable fixation of the needle on the syringe body.

Preferably, the envelope-forming part, the fixation piece and the divisible portions are made of the same component, the divisible portions consisting of zones having reduced dimensions limited by openings made in the said component.

Also preferably, the openings have contours such as after dividing the divisible portions, the part of the divisible portions remaining connected to the fixation piece forms projections capable of cooperating, in rotation around the longitudinal axis of the cap, with the part of the divisible portions remaining connected to the envelope-forming part also in projection to make possible the unscrewing of the fixation portion in relation to the body of the syringe.

It is understood that the periphery of the openings, which define the divisible portions, is defined in such a way that, after separation of the envelope-forming part in relation to the fixation piece, the part of the divisible portions remaining connected to the envelope-forming part may be used as a sort of screwdriver, these projecting parts cooperating with the part of the divisible portions remaining connected to the fixation piece.

Also preferably, the internal surface of the envelope-forming part comprises reliefs capable of cooperating in rotation with reliefs arranged in the base of the needle, when the projecting parts of the divisible portions remaining connected to the fixation piece and to the envelope-forming part cooperate in rotation.

It is understood that the reliefs made in the internal surface of the envelope-forming part are able to cooperate with corresponding reliefs arranged in the base of the needle when the projecting parts of the divisible portions remaining connected to the fixation piece and the envelope-forming part cooperate in rotation for performing the unscrewing of the fixation piece in relation to the syringe body. This rotation of the envelope-forming part simultaneously makes possible the unscrewing of the needle due to cooperation of the reliefs arranged in the base of the needle and the reliefs arranged in the internal surface of the envelope-forming part.

A second object of the present invention is to provide a syringe of the type described above and equipped with a cap of the type also defined above.

According to the present invention, the syringe comprises:
a syringe body having a fixation end provided with an internal screw thread;
a needle assembly comprising a needle and a base, the said base comprising two radial extensions capable of cooperating with the said internal screw thread of the syringe body; and
a needle cap;
the said syringe is characterized in that the said needle cap comprises:
an envelope-forming part for receiving the needle proper;
a fixation piece capable of receiving the base of the needle and comprising on its external surface a threaded section capable of cooperating with an internal screw thread arranged at one end of the syringe body; and
divisible portions for connecting the said envelope-forming part and the said fixation piece, the divisible portions having certain dimensions so that they break when a predetermined torque is applied to the envelope-forming part, even though the threaded section of the fixation piece was engaged with the internal screw thread of the syringe body, the fixation piece thus forming in the internal screw thread of the syringe body a locknut for the radial extensions of the base of the needle assembly which are also engaged in the internal screw thread.

Other features and advantages of the present invention shall become more evident after reading the description below of preferred embodiments of the present invention given by way of nonlimiting examples. The description refers to the attached figures, in which:

FIG. 1, already described, shows a longitudinal section of a syringe body;

FIG. 1A shows the detail of the end of the syringe body on which the needle has to be fixed;

FIG. 2, already described, shows a needle assembly that can be used in the present invention;

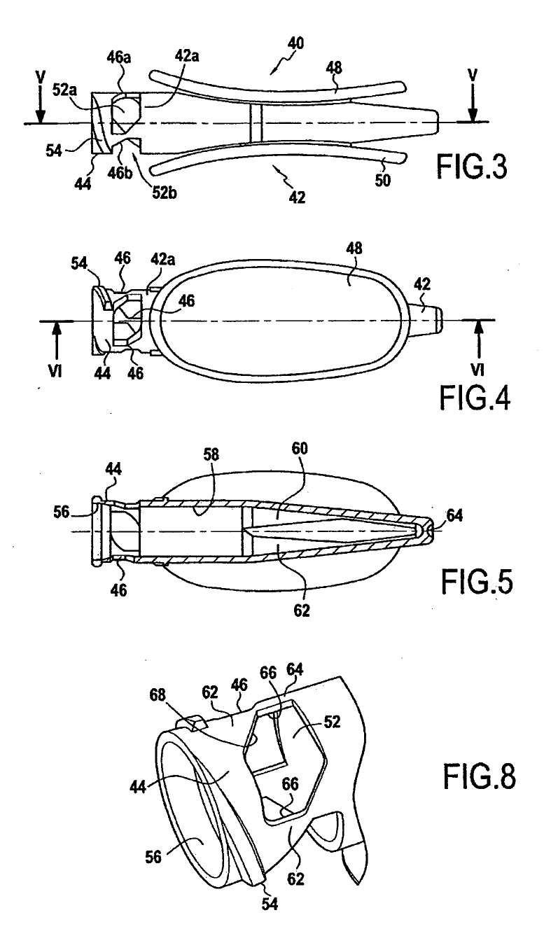

FIG. 3 is a lateral view of the needle cap;

FIG. 4 is a top view of the needle cap;
FIG. 5 is a sectional view along line V-V of FIG. 3;

FIG. 6 is a longitudinal sectional view along line VI-VI of FIG. 4;

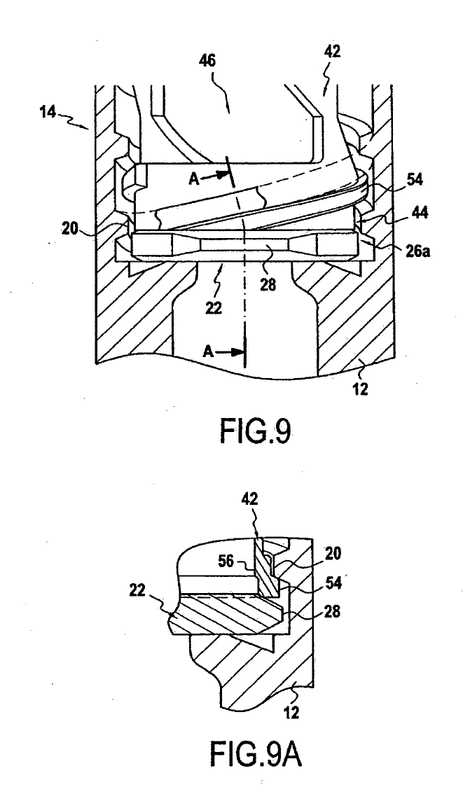

FIG. 7 is a cross-sectional view along line VII-VII of FIG. 6;
FIG. 8 is a perspective view of the end of the needle cap;
FIG. 9 is a partial longitudinal sectional view showing the fixation of the needle and of the cap on the syringe body; and
FIG. 9A is a detail view along line A-A of FIG. 9.

As FIG. 3 shows, the cap 40 comprises an envelope-forming part 42, a fixation piece 44 on the syringe body and divisible portions such as 46a, 46b, which connect the end 42a of the envelope-forming part to the fixation piece 44. The envelope-forming part 42 is preferably equipped with two external gripping surfaces 48 and 50.

Preferably, the cap is produced by molding. The various components making up the cap, i.e., the envelope-forming part 42, the fixation piece 44 and the divisible portions 46a, 46b are made from a same assembly. The divisible portions 46a, 46b are defined by openings such as 52a, 52b. These openings also define the end 42a of the envelope-forming part 42 and one of the ends of the fixation piece 44. The fixation piece 44 is provided on its external surface with a threaded section 54 capable of cooperating with the internal screw thread 20 of the ferrule 18 of the syringe body. As is better shown in FIG. 5, the fixation piece 44 has an axial passage 56, which has a slightly oval shape in cross section for cooperating with the base 26 of the needle assembly 22. The axial passage 56 ensures a certain clamping of the base 26 of the needle without, nevertheless, creating an effective interlock.

Two ribs in relief form 60 and 62 arranged between the central part of the envelope-forming part 42 and its closed end 64 are provided on the internal surface 58 of the envelope-forming part.

These internal ribs 60 and 62 are capable of cooperating with the fins 32 and 34 of the base 26 of the needle assembly as will be explained later.

The particular shape of the contour of the openings 52 which define the divisible portions 46 shall be described in greater detail by referring to FIG. 8. Each divisible portion 46 comprises a part 62 which remains connected to the fixation piece 44 and a part 64 which remains connected to the envelope-forming part 42 after dividing of the divisible portions. Each of the portions 64 and 62 comprises a side that is more or less parallel to the longitudinal axis of the cap and that has the reference number 66 and an inclined side 68. It is understood that, after dividing of the divisible portions, the parts 62 and 64 of these divisible portions shall be able to be contacted by their linear edge[s] 66 to make possible an unscrewing of the fixation piece 44 in relation to the syringe body, the cap or more precisely the envelope-forming part of the cap being used as the key for unscrewing the fixation piece 44.

The mode of use of the cap, which is the object of the present invention, shall be explained now. At first, the needle assembly 22 is put in place in the cap 40 in such a way that the part 26a of the base 26 of the needle assembly is engaged in the axial opening 56 of the fixation piece 44 ensuring a temporary connection between the cap and the needle assembly. The extensions 28 and 30 of the needle assembly are arranged outside of the fixation piece 44. This assembly, which may be held by the grip components 48 and 50, is engaged on the end of the syringe body 12. The truncated cone part 16 of the syringe body penetrates the axial passage of the base 26 of the needle assembly. At first, the extensions 28 and 30 of the base of the needle assembly cooperate with the internal screw thread 20 of the ferrule 18 of the syringe body. In a later phase, the threaded section 54 of the fixation piece 44 of the cap will also come into contact with the internal screw thread 20. The user continues to bring about the rotation of the cap in relation to the syringe body. Then, the base 26 of the needle assembly is blocked in translation by the truncated cone part 16. The continuation of the rotation of the cap brings about a torque of clamping the fixation piece 44 in the internal screw thread 20. When the predetermined clamping torque is reached, the divisible portions 46 are broken and the envelope-forming part is separated from the fixation piece. This part 42 may be removed. It is understood that the fixation piece 44, which is screwed in the internal screw thread 20, constitutes a locknut for the extensions 28 and 30 of the base 26 of the needle assembly 22. The needle 24 is thus solidly and effectively fixed at the end of the syringe body 12.

FIGS. 9 and 9A show detail views of the interlock of the needle 24 by the fixation piece 44 of the cap on the syringe body. At the end of the screwing operation, the triangular shape of the engaging end 54a of the threaded section 54 of the fixation piece 44 will be wedged, due to a wedge effect, between the internal screw thread 20 of the syringe body and a radial extension 28 of the base 26 of the needle. A highly effective fixation of the needle on the syringe body is thus obtained.

When one wants to proceed with removing the needle in relation to the syringe body, the envelope-forming part 42 is used. More precisely, the portions 64 of the divisible portions, which have remained integral with the envelope-forming part between the portions 62 which have remained integral with the fixation piece 44, are engaged. By turning the envelope-forming part, the linear surfaces 66 of the respective portions will come into rotating contact. Moreover, in this relative position of the fixation piece and of the envelope-forming part, the fins 32 and 34 of the base of the needle will cooperate with the ribs 60 and 62 arranged inside the envelope-forming part 42. The rotation of the envelope-forming part therefore simultaneously leads to the rotation of the fixation piece 44 and that of the base 26 of the needle assembly. Thus, easy removal of the needle in relation to the syringe body 12 is obtained.

1. Cap for a syringe needle designed to be mounted on the body of a syringe, the cap comprising:
an envelope-forming part for receiving the needle;
a fixation piece capable of receiving the base of the needle and comprising on its external surface a threaded section capable of cooperating with an internal screw thread arranged at one end of the syringe body; and
divisible portions for connecting the said envelope-forming part and the said fixation piece, the divisible portions having certain dimensions so that they break when a predetermined torque is applied to the envelope-forming part, even though the threaded section of the fixation piece was engaged with the internal screw thread of the syringe body.

2. The cap in accordance with claim 1 wherein said fixation piece has a central cutout capable of cooperating along the longitudinal axis of the cap with the external surface of the base of the needle.

3. The cap in accordance with claim 1 wherein the envelope-forming part, the fixation piece and the divisible parts are made of the same component, the divisible portions being made up of zones of reduced dimensions limited by openings made in the said component.

4. The cap in accordance with claim 3 wherein the openings have contours such as after division of the divisible portions, the part of the divisible portions remaining connected to the fixation piece forming projections capable of cooperating, in rotation around the longitudinal axis of the cap, with the part of the divisible portions remaining connected to the envelope-forming part also as a projection for making possible the unscrewing of the fixation portion in relation to the body of the syringe.

5. The cap in accordance with claim 4, wherein the internal surface of the envelope-forming part comprises reliefs capable of cooperating, in rotation, with reliefs arranged in the base of the needle, when the projecting parts of the divisible portions remaining connected to the fixation piece and to the envelope-forming part cooperate in rotation.

6. Syringe comprising:
a syringe body having a fixation end provided with an internal screw thread;
a needle assembly comprising a needle and a base, the said base comprising two radial extensions capable of cooperating with the said internal screw thread of the syringe body; and
a needle cap comprising:
an envelope-forming part for receiving the needle proper;
a fixation piece capable of receiving the base of the needle and comprising on its external surface a threaded section capable of cooperating with an internal screw thread arranged at one end of the syringe body; and
divisible portions for connecting the said envelope-forming part and the said fixation piece, the divisible portions having certain dimensions so that they break when a predetermined torque is applied to the envelope-forming part, even though the threaded section of the fixation piece was engaged with the internal screw thread of the syringe body, the fixation piece thus forming in the internal screw thread of the syringe body a locknut for the radial extensions of the base of the needle assembly which are also engaged in the internal screw thread.

7. The syringe in accordance with claim 6 wherein said fixation piece has a central cutout capable of cooperating along the longitudinal axis with the external surface of the base of the needle.

8. The syringe in accordance with claim 6 wherein the envelope-forming part, the fixation piece and the divisible portions are made of the same component, the divisible portions being made up of zones having reduced dimensions limited by openings in the component.

9. The syringe in accordance with claim 8, wherein the openings have contours such as after dividing the divisible portions, the part of the divisible portions remaining connected to the fixation piece forms projections capable of cooperating, in rotation around the longitudinal axis of the cap, with the part of the divisible portions remaining connected to the envelope-forming part also projecting to make possible the unscrewing of the fixation portion in relation to the body of the syringe.

10. The syringe in accordance with claim 9, wherein the internal surface of the envelope-forming part comprises reliefs capable of cooperating, in rotation, with reliefs arranged in the base of the needle, when the projecting parts of the divisible portions remaining connected to the fixation piece and to the envelope-forming part cooperate in rotation.

11. The syringe in accordance with claim 6 wherein the portion of the base of the needle having the radial extensions projects out of the fixation piece when the needle assembly is engaged in the cap.

12. The syringe in accordance with claim 6 wherein the engaging end of the threaded section of the fixation piece produces a wedge effect, between the internal screw thread of the syringe body and a radial extension of the base of the needle assembly.

13. The cap in accordance with claim 2 wherein the envelope-forming part, the fixation piece and the divisible parts are made of the same component, the divisible portions being made up of zones of reduced dimensions limited by openings made in the component.

14. The syringe in accordance with claim 7 wherein the envelope-forming part, the fixation piece and the divisible portions are made of the same component, the divisible portions being made up of zones having reduced dimensions limited by openings in the component.

15. The syringe in accordance with claim 7 wherein the portion of the base of the needle having the radial extensions projects out of the fixation piece when the needle assembly is engaged in the cap.

16. The syringe in accordance with claim 8 wherein the portion of the base of the needle having the radial extensions projects out of the fixation piece when the needle assembly is engaged in the cap.

17. The syringe in accordance with claim 9 wherein the portion of the base of the needle having the radial extensions projects out of the fixation piece when the needle assembly is engaged in the cap.

18. The syringe in accordance with claim 10 wherein the portion of the base of the needle having the radial extensions projects out of the fixation piece when the needle assembly is engaged in the cap.

19. The syringe in accordance with claim 7 wherein the engaging end of the threaded section of the fixation piece produces a wedge effect between the internal screw thread of the syringe body and a radial extension of the base of the needle assembly.

20. The syringe in accordance with claim 8 wherein the engaging end of the threaded section of the fixation piece produces a wedge effect between the internal screw thread of the syringe body and a radial extension of the base of the needle assembly.
